(19) United States
(12) Patent Application Publication
(10) Pub. No.: US 2018/0132519 A1
(43) Pub. Date: May 17, 2018

(54) ANIMAL WEIGHT MANAGEMENT

(71) Applicant: HEYREX LIMITED, Wellington (NZ)

(72) Inventor: MARK CAMERON SOLLY, Wellington (NZ)

(21) Appl. No.: 15/575,557

(22) PCT Filed: May 20, 2016
(86) PCT No.: PCT/NZ2016/050084

§ 371 Date: Nov. 20, 2017

(30) Foreign Application Priority Data
May 21, 2015 (NZ) 708354

(51) Int. Cl. A23L 33/00; A01K 29/00; A61B 5/00

(52) U.S. Cl. CPC A23L 33/30; A01K 29/005; A01K 27/009; A61B 5/4866; A61B 5/4833

(57) ABSTRACT

A system for animal weight management relates to the technical field of optimising animal weight and therefore health. It can be a problem that domestic animals may become underweight or overweight and it can be difficult to obtain accurate information relating to diet and exercise and so difficult to effectively treat animals. The system comprises a database having animal food information, a food plan section to allow creation of a food plan for an animal, an exercise plan section to allow creation of an exercise plan for the animal, a monitoring section including storage to store information about the weight history of the animal and the animal's activity, and a monitor adapted to be attached to the animal and provide information about the animal's activity to the monitoring section. The system is principally used by animal health professionals and animal caregivers on domestic animals to optimise the weight and therefore health of the animals.

### FIELD OF INVENTION

The invention relates to a method and system for assisting in animal weight management.

### BACKGROUND

Animals need a balance of diet and exercise to maintain a healthy weight. Many domestic animals are not getting the right balance and as a result become underweight, or, more commonly, overweight. It is important for the long term health of an animal that it maintains a healthy weight.

For domestic animals diet and exercise are the responsibility of the animal's owner and/or caregiver. If a domestic animal becomes overweight its owner may take it to a vet or other animal healthcare professional. Typically the vet or animal healthcare professional will set a diet for the animal and monitor the animal's weight over time to try to achieve a healthy weight for the animal. This system generally relies on the owner/caregiver of the animal providing the animal healthcare professional with an accurate account of the animal's eating and exercise. However, many animal owners will not be entirely accurate with this information. The owner may underestimate the amount of snacks that the animal is consuming and overestimate the amount of exercise that the animal is receiving. This makes it difficult for the animal health professional to estimate how many calories the animal is consuming and how many calories the animal is burning.

It is an object of the present invention to go at least part way towards providing an animal weight management system and method. It is a further object to provide the public with a useful choice.

The term “comprising”, if and when used in this document, should be interpreted non-exclusively. For example if used in relation to a combination of features it should not be taken as precluding the option of there being further unnamed features.

### SUMMARY OF THE INVENTION

In broad terms in one aspect the invention comprises a system for animal weight management comprising:
a database comprising animal food information,
a food plan section to allow creation of a food plan for an animal,
an exercise plan section to allow creation of an exercise plan for the animal,
a monitoring section including storage to store information about the weight history of the animal and the animal's activity, and
a monitor adapted to be attached to the animal and provide information about the animal's activity to the monitoring section.

In broad terms in another aspect the invention comprises a method of animal weight management comprising the steps of:
generating a food plan for an animal,
generating an exercise plan for the animal,
providing the food and exercise plans to an owner or caregiver of the animal,
monitoring the energy expended by the animal using a monitor on the animal,
comparing energy expended by the animal to energy consumed by the animal, and
varying the food and/or exercise plans in response to the comparison of energy expended and energy consumed by the animal.

The energy expended and energy consumed by the animal may be determined as calories expended and consumed or metabolizable energy or in any other suitable format.

In one embodiment the food plan is selected from a number of food plans stored in a database.

Optionally the exercise plan is stored in a database.

Optionally the food plan is stored in a database.

### DRAWINGS

The invention will be described in more detail with reference to the accompanying drawings in which:

FIG. 1 is a flow chart showing the steps of the system;

FIG. 2 shows an image of a screen showing an exercise plan and eating plan for an animal; and

FIG. 3 is a flow chart of weight management dataflow and processes.

### DETAILED DESCRIPTION

Some preferred forms of the invention will now be described by way of example. It should be understood that these are not intended to limit the scope of the invention but rather to illustrate optional embodiments.

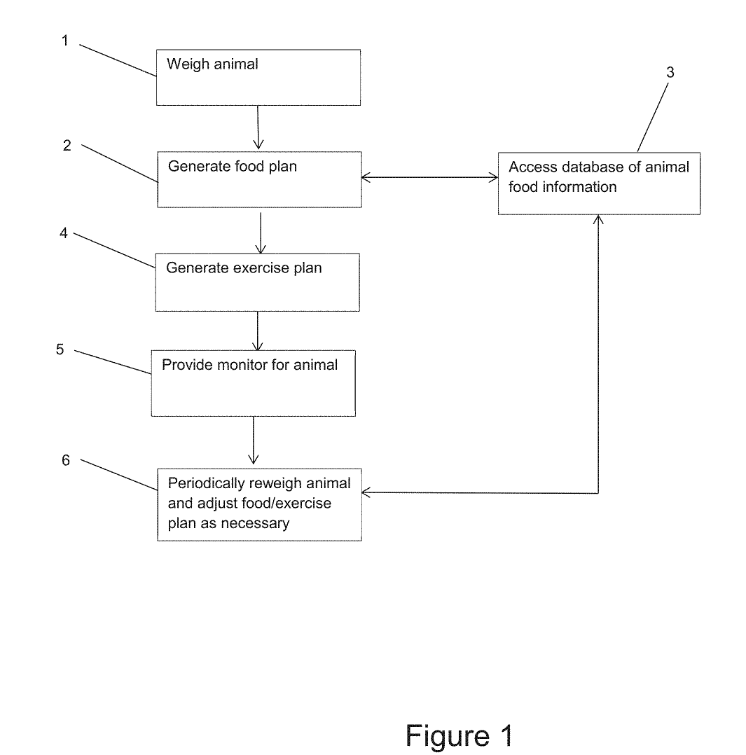

FIG. 1 is a flow chart showing steps of the system. When an animal with a weight management problem is assessed by an animal health professional one of the first steps is to determine the weight of the animal at step 1. This can be done directly by weighing the animal or by accessing information on the animal's weight. For example, the animal may have been weighed recently and the weight may be accessible to the animal health professional.

The animal health professional creates a food plan for the animal at step 2. The food plan describes the amount of particular foods for the animal to eat each day. The food plan may list more than one food and will provide amounts of the food. The animal health professional accesses a database of animal food information 3 to assist in creating the food plan 2. The database of animal food includes information about standard foods and treat foods. The information may include calorie content, wet vs dry food and nutrition information. The animal health professional can select one or more foods to provide the animal with its nutritional requirements. The animal health professional can select the food and daily (weekly or other frequency) quantity of each food to be fed to the animal. When the food is selected the program accesses the food database and calculates the number of calories in the food (or the metabolizable energy provided by the food) at the quantity specified. The animal health professional can adjust the amount of food to reach a goal number of daily calories (or goal amount of metabolizable energy) for the animal. Alternatively the goal number of calories could be expressed as a weekly (or other frequency) goal. Any planned calorie intake for which a listing in the database does not exist may also be added to the food plan. Food may be ordered via the system based on the diet set by the animal health professional or other user.

The animal health professional also generates an exercise plan for the animal at step 4. The exercise plan will depend on the species of animal, general health of the animal and any medical conditions experienced by the animal. Additionally the breed, gender, age and reproductive status of the animal may also be considered when generating an exercise plan for the animal. The animal's medical records may be accessible to the animal health professional.

It should be noted that the order in which the food and exercise plans are generated is not important. The animal health professional may generate the exercise plan before the food plan or vice versa. The two plans may be developed at the same time.

Both the exercise plan and food plan are provided to the animal's owner or caregiver. The animal's owner or caregiver is also provided with a monitor at step 5 to monitor the animal. The monitor may monitor the animal's experiential temperature and activity. Data from the monitor is returned to the system. One preferred form of monitor and monitor system is described in more detail below.

Periodically the animal's progress is reviewed 6. Data from the monitor is analysed to determine how many calories the animal is burning (or how much energy the animal is expending) and the animal is reweighed. If the animal's weight is moving in the right direction (decreasing if the animal is overweight and increasing if the animal is underweight) then the exercise and food plans may not need adjusting. If the animal is not losing or gaining weight at a rate that would be expected given the animal's food and exercise plan then one of both of these plans may need adjusting. When adjusting the animal's food plan the animal health professional may refer back to the database of animal food information. Alternatively the animal health professional may review the data from the animal's monitor and determine whether the animal's owner/carer is following the exercise and/or food plan. For example if the animal is to receive two meals a day but the animal's activity shows that it is eating two meals and lots of snacks then the animal health professional can discuss this with the animal's owner/carer. If the animal's activity shows that the animal is not getting all the exercise specified on the exercise plan this can also be discussed with the animal's owner/carer. Using the monitor provides a more accurate assessment of the animal's activity than relying on the animal's owner/carer for information.

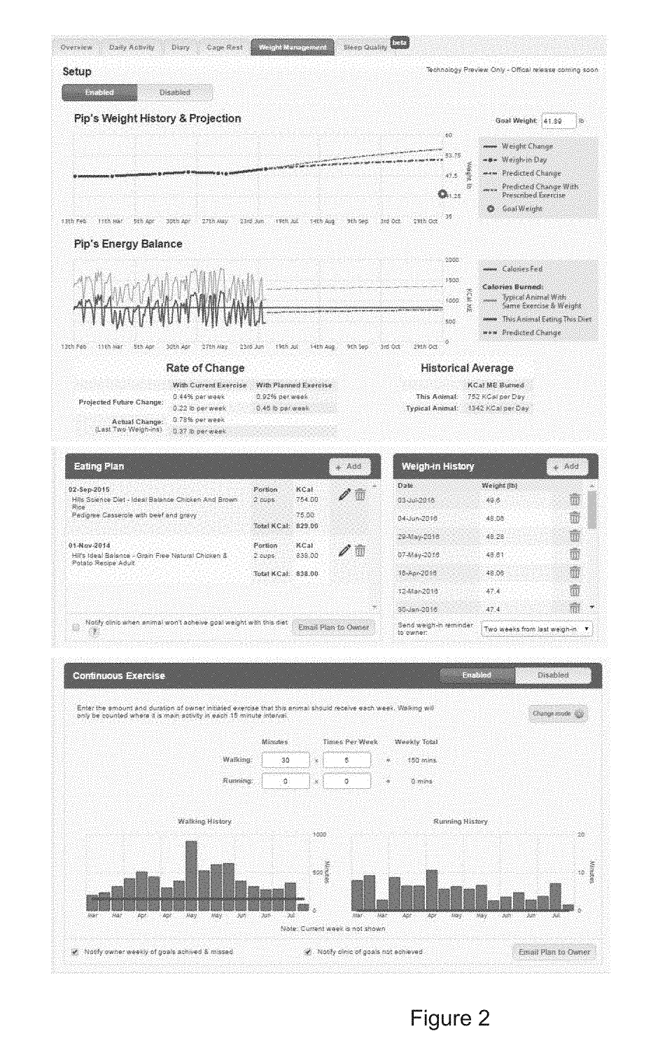

FIG. 2 is a screen shot showing an example exercise plan and an example food plan for an animal. FIG. 2 also shows an example weigh-in history, weight history and projection and energy balance data for the animal. In preferred embodiments the program display is a selection of tables and/or graphs that allow an animal health professional to select what parts of the service they wish to use and when animal care goals they wish to view.

The example exercise plan shown at the bottom left of FIG. 2 includes both walking and running. The program operator (typically an animal healthcare professional) can select the duration of a number of different types of exercise and the frequency of each type of exercise. In the plan shown in FIG. 2 walking has been selected for 15 minutes duration, 3 times per week. Running has been selected for 3 minutes 3 times per week. The time given for different types of exercise could be entered manually, selected from a drop down menu or provided in any other suitable way. Weekly totals for running and walking are given. A history of both types of exercise is given below the exercise selection. The history can be provided by data from the monitor worn by the animal or by reporting from the animal's owner/carer.

The history will show any changes made to the exercise plan. FIG. 2 shows the exercise history as a line and bar graph. The line represents the planned exercise and the bars represent the actual exercise performed by the animal. In this format it is easy to see where the animal's actual exercise is in relation to the planned exercise. In the example shown in FIG. 2, the animal has usually completed the walking part of the exercise plan but has only once completed the running part of the exercise plan. Any other suitable method to display the animal's exercise could be used. For example, a line graph could be used.

The exercise plan shown is suitable for a domestic animal like a dog. Different types of exercises may be selectable for different animals. For example, jumping, agility training, walking, running, swimming, and/or climbing may be selectable. The animal health professional selects the most appropriate exercise or exercises for the animal.

A weigh-in history may also be shown (see the middle right of FIG. 2). This history shows the animal's weight in chronological order (either ascending or descending). Alternatively the weight of the animal could be displayed on a graph. Further graphs may show the weight history for the animal in graphical form over time and the animal's energy balance as shown in FIG. 2. The weight graph shown in FIG. 2 also includes markers showing the days on which the animal was weighed, the predicted change in weight of the animal, the predicted change in weight of the animal if the animal completed the prescribed exercise and the goal weight for the animal. The energy balance graph may show the calories burned by the animal, the calories fed to the animal, the calories burned by a typical animal with the same exercise and weight and the predicted change over time.

Another section of the display shown in FIG. 2 is the alerts (shown at the bottom right). These may be sent by email to the owner/carer of the animal. The alerts can be selected by the animal health professional. These alerts include (but are not necessarily limited to):
Automatically calculating portions based on exercise based on exercise and notify the owner weekly,
Notify a clinic when the animal's diet requires a review due to an extended change in the amount of calories burned,
Sending a monthly (or any other selected frequency) weigh-in reminder to the animal's owner/carer,
Notifying the owner/carer of weekly exercise goals and success, and
Notifying an animal health clinic with responsibility for the animal's health of any goals not achieved.

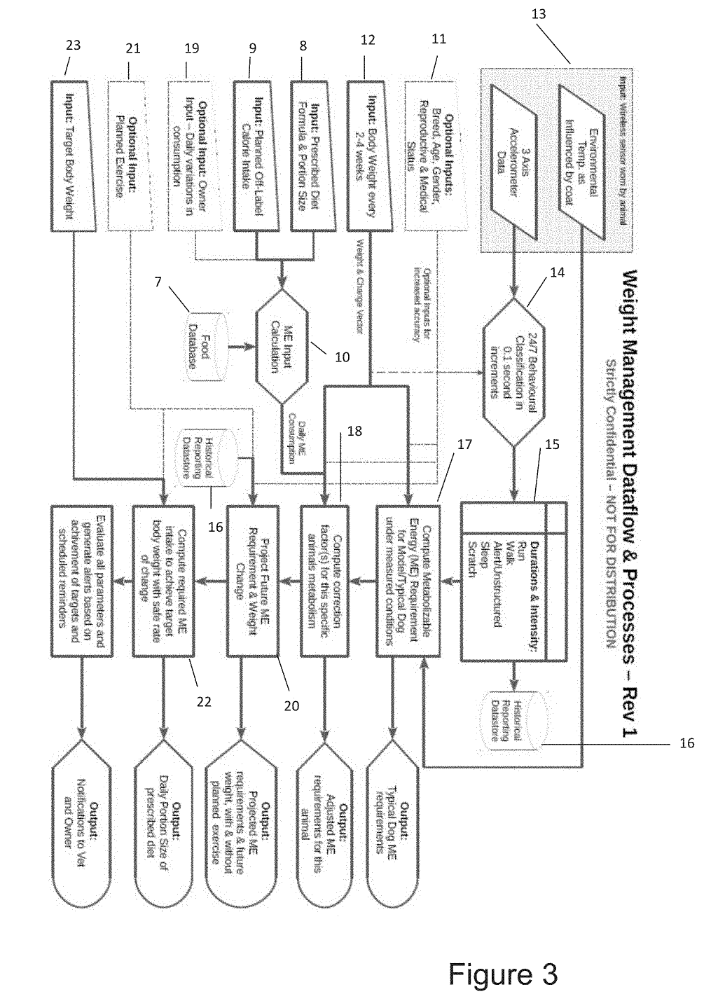

FIG. 3 shows the weight management data flow and processes.

The food plan is linked to a food database 7 containing animal food information. The animal food information includes content details of the food including calorie volumes, ingredients, wet vs dry food etc. The animal food database 7 contains information about standard foods and treat foods. This information includes information about the metabolizable energy and/or calories provided by each food. The animal health professional can select one or more foods to provide the animal with its nutritional requirements and create a prescribed food plan 8. The animal health professional can select the food and daily quantity of each food to be fed to the animal. Additionally planned off-label calorie intake 9 can be added to the weight management system. When the food and planned additional calorie intake 9 are provided, the program accesses the food database 7 and calculates the number of calories and/or metabolizable energy in the food at the quantity specified 10. The animal health professional can adjust the amount of food to reach a goal number of daily calories for the animal. Alternatively or additionally the metabolizable energy of the food is provided and the animal health professional can adjust the amount of food to reach a goal metabolizable energy for the animal.

The program can also be used to promote certain food brands or specific diet foods.

In generating the food plan the animal health professional can take into account the animal's general health based on the animal's medical records, needs and certain care objectives 11. In some embodiments medical records for the animal may be stored in a database accessible to the system (or part of the system) so that the animal health professional can access the medical records for the animal when generating food and exercise plans.

While the animal is being cared for using the program the animal's weight is monitored and recorded in a database 12. For example, the animal may be weighed with a frequency between 2 and 4 weeks. The frequency at which the animal is weighed may change over time. The weight of the animal over time can be shown to the animal health professional as shown in FIG. 2. This lets the animal health professional monitor the animal's weight and determine whether the food and/or exercise plan requires adjustment.

As part of the plan the animal is provided with a monitor that collects activity and experiential temperature data. From the activity and experiential temperature data the system can determine how many calories the animal is burning. The monitor may be a monitor like the one described in U.S. Pat. No. 8,866,605 to Gibson et al which is herein incorporated by reference. The monitor described in U.S. Pat. No. 8,866,605 uses non-invasive on-animal sensors to continuously and automatically observer and monitor an animal's behaviour and use this information to assess and detect changes in wellbeing. The data can be made available to animal health professionals in a convenient way without the need to be in close contact with the animal. The data logged by the data-logger as part of the monitor on the animal may include movement (for example, via accelerometers), vibration, tilt, location (including altitude), sound, experiential temperature, light level, humidity, heart rate, breathing rate, odour, time and date, weight, food consumed and water consumed.

In some embodiments the data logger of the monitor is embedded in a collar worn by the animal. In preferred embodiments the monitor uses a wireless transceiver to communicate with a data relay separate to the monitor. The data relay must be located within range of the monitor at predetermined or intermittent times so that data from the monitor can be downloaded into the data relay. This means that the animal can be away from the data relay for periods of time (for example, while exercising). When the animal (and its monitor) comes back into range of the data relay and data stored in the monitor is downloaded into the data relay.

The data relay receives data from the monitor and relays the data to a data storage means 13 accessible by the animal health professional. The data may be relayed to the data storage means 13 by any suitable means, including by the internet or mobile phone network.

The data storage means is preferably a computer database server. The computer database server may operate as a central location for the storage, retrieval, processing and display of data from many data logger units. The collected data is processed and stored. Processing the data includes analysing the data with a computer processor to determine from the data the activities of the animal 14,15. The activities that can be determined include the physical activity of the animal, such as how much the animal is moving around and how many calories the animal is burning. Data from a three axis accelerometer in the monitor may be classified into a behavioural classification in small periods. In one embodiment the behavioural classification may be in 0.1 second increments 14. Alternatively other increments can be used. The data can then be used to determine the duration and intensity of typical behaviours such as running, walking, alert/unstructured, sleeping, scratching etc. 15. Body weight data can be used in the behavioural classification. The behaviour information can be stored in a database 16.

The data is accessible to the rest of the weight management program. In the weight management program the data may be displayed in any suitable form. For example, raw data could be displayed. The behavioural data 15 can be combined with experiential temperature data from the animal monitor and the last known weight of the animal 12 to compute the metabolizable energy requirement for a typical animal measured under normal conditions 17. Alternatively a daily activity graph of the animal could be displayed showing when the animal is sleeping, doing mild exercise, doing strenuous exercise etc. The data from the monitor may allow the animal health professional to determine whether the animal is meeting the exercises specified in the exercise plan. For example, if an exercise plan specifies 30 minutes walking for the animal every day the animal health professional should be able to see this activity from the animal monitor data. If the animal health professional cannot see this activity the animal health professional can discuss this with the animal's owner/carer.

If an animal is overweight, one aim of the exercise plan is to burn off calories that the animal has consumed or excess calories stored as fat on the animal. The data available to the weight management program preferably shows how many calories the animal is burning in the intervals between seeing the animal health professional and/or being monitored by the animal health professional. This data may be displayed in any suitable format, for example daily or weekly calories burned either as a number or a graph (see for example the graph in FIG. 2). The animal health professional can compare the number of calories burned with the activity levels of the animal and adjust the exercise plan if necessary to assist the animal in reaching the target number of calories burned. In preferred embodiments the program will compare calories consumed and calories burned and can recommend changes in the calories to be consumed (and hence food plan) based on progressive results. These changes could be an increase or a decrease in calories consumed.

In another embodiment the metabolizable energy requirements of the animal may be determined 17. Metabolizable energy is the net energy from a food available to an animal after the utilization of some energy in the process of digestion and absorption and the loss of some material as being undigested or indigestible. The metabolizable energy requirement for a typical animal with the same body weight and activity patterns as the animal being monitored can be computed using the animal's body weight and activity pattern from the monitor as well as the animal's environmental temperature. The metabolizable energy that the animal is receiving 18 can be computed using the food plan for the animal 8 and any planned off-label calorie intake 9 at step 10. Using the food database 7 the metabolizable energy of the food plan can be calculated at step 10. The daily metabolizable energy consumption for the animal 10 can be used, along with the animal's body weight 12 and computed metabolizable energy for a typical animal with the animal's weight and behavioural patterns 17 to determine the metabolizable energy requirements for the actual animal 18. If the metabolizable energy requirements for the animal exceed that daily metabolizable energy consumed by the animal, it can be expected that the animal will lose weight over time. Conversely, if the metabolizable energy requirements for the animal are less than the metabolizable energy consumed by the animal, it can be expected that the animal will gain weight over time.

In addition to the food plan, the animal's owner/carer can discuss with the animal health professional any daily variations in food consumption 19. For example, the animal may have not eaten one or more meals during the week. Alternatively, the animal may have eaten additional food. Variations in the food plan can be included in the metabolizable energy calculation 10 for the animal.

The future metabolizable energy requirements of the animal 20 can be projected using the current metabolizable energy requirements of the animal 18 and the animal's history 16 (stored in a database). Any additional planned exercise 21 can be used in calculating the future metabolizable energy requirements of the animal. The projected weight of the animal 20 can also be computed based on the projected metabolizable energy requirements, exercise plan and any additional planned exercise. The projected metabolizable energy requirements can be computed and displayed along with the projected weight both with and without an additional exercise.

The required metabolizable energy intake 22 to achieve the animal's target weight 23 can be computed using the target weight of the animal and the projected further metabolizable

The program displaying the data is accessible to the animal health professional. It may also be accessible (by user name and password) to the animal's owner. The animal's owner may be able to access data relating to their animal but not be able to adjust the exercise and food plans. In some embodiments that animal health professional may be able to print a report of the animal's progress for the animal's owner or electronically send the report to the animal's owner.

In use an animal with a weight management problem (either overweight or underweight) may be taken to an animal health professional. The animal health professional provides a food plan for the animal using foods selected from a database. The food plan specifies the brand of food, amount of food per day and provides the animal health professional with the number of calories provided by the food in the food plan. The animal health professional also provides an exercise plan. The initial exercise plan may be based on the amount of exercise the animal is currently getting as described by the animal's owner or carer. The animal health professional weighs the animal and the animal's weight is included in the program. A note of the animal's goal weight may be recorded. The animal health professional may weigh the animal before deciding on a food an exercise plan for the animal and may decide on the food an exercise plans based in part on the animal's current weight. The animal is fitted with an activity monitor. The food and exercise plans are provided to the animal's owner.

Over time the animal health professional reviews the progress the animal is making towards losing or gaining weight. The animal health professional may see and weigh the animal at intervals. At these times the animal health professional may also review the animal's activity levels and the animal's food plan. If the is losing or gaining weight at a slower than anticipated rate the animal health professional may adjust the animal's food and/or exercise plans. Adjustment of the animal's food and/or exercise plans may also be needed if the animal is losing or gaining weight at a greater than anticipated rate.

The animal health professional may also review the animal's progress at any time without weighing the animal or the need for the animal to be present. The animal health professional can access the food plan and up-to-date information about the number of calories that the animal is burning from the animal's monitor. The animal health professional can then adjust the food and/or exercise plan and advise the animal's owner of any adjustments.

While some preferred aspects of the invention have been described by way of example it should be appreciated that modifications and improvements can occur without departing from the scope of the invention.

1. A system for animal weight management comprising:
a database comprising animal food information,
a food plan section to allow creation of a food plan for an animal,
an exercise plan section to allow creation of an exercise plan for the animal,
a monitoring section including storage to store information about the weight history of the animal and the animal's activity, and
a monitor adapted to be attached to the animal and provide information about the animal's activity to the monitoring section.

2. A system according to claim 1, wherein energy expended and energy consumed by the animal may be determined as calories expended and consumed or metabolizable energy or in any other suitable format.

3. A system according to claim 1, wherein the food plan is selected from a number of food plans stored in a database.

4. A system according to claim 1, wherein the exercise plan is stored in a database.

5. A system according to claim 1, wherein the created food plan is stored in a database.

6. A method of animal weight management comprising the steps of:
generating a food plan for an animal,
generating an exercise plan for the animal,
providing the food and exercise plans to an owner or caregiver of the animal,
monitoring the energy expended by the animal using a monitor on the animal,
comparing energy expended by the animal to energy consumed by the animal, and
varying the food and/or exercise plans in response to the comparison of energy expended and energy consumed by the animal.

7. A method according to claim 6, wherein the step of monitoring energy expended and energy consumed by the animal comprises determining the calories expended and consumed by the animal or metabolizable energy received and/or expended by the animal.

8. A method according to claim 6, wherein the step of generating the food plan comprises selecting a food plan from a number of food plans stored in a database.

9. A method according to claim 6, further including the step of storing the generated food plan in a database.

10. A method according to claim 6, wherein the food plan is stored in a database.

11. (canceled)

12. (canceled)

13. (canceled)

14. (canceled)
